(19) United States
(12) Patent Application Publication
(10) Pub. No.: US 2019/0099520 A1
(43) Pub. Date: Apr. 4, 2019

(54) REINFORCED TISSUE MATRICES

(71) Applicant: LifeCell Corporation, Madison, NJ (US)

(72) Inventors: Israel Jessop, Annandale, NJ (US); Hui Xu, Plainsboro, NJ (US); Hui Li, Cranbury, NJ (US); Nathaniel Bachrach, Clifton, NJ (US)

(21) Appl. No.: 16/150,387

(22) Filed: Oct. 3, 2018

Related U.S. Application Data
(60) Provisional application No. 62/567,376, filed on Oct. 3, 2017.

(51) Int. Cl. A61L 27/36; A61K 35/32; A61L 27/26

(52) U.S. Cl. CPC A61L 27/3633; A61K 38/012; A61L 27/26; A61K 35/32

(57) ABSTRACT

Devices, methods of producing devices, and methods of treating an anatomic defect are provided. The devices and methods can include a flexible sheet of acellular tissue matrix and one or more elongated synthetic elements. The one or more synthetic elements may extend through the flexible sheet such that the one or more synthetic elements extend substantially parallel to a first top surface and a second bottom surface within one or more portions of the flexible sheet, and may be exposed on one or more portions of the first top surface of the device.

This application claims priority to U.S. Provisional Patent Application 62/567,376, which was filed on Oct. 3, 2017, and which is incorporated by reference in its entirety.

The present disclosure relates to tissue treatment devices, more particularly to tissue matrices combined with elongated synthetic elements to provide structural reinforcement, and to methods for treatment of anatomic defects with tissue treatment devices and to methods for making tissue treatment devices.

Various tissue-derived products are used to regenerate, repair, or otherwise treat diseased or damaged tissues and organs. Such products can include tissue grafts and/or processed tissues (e.g., acellular tissue matrices from skin, intestine, or other tissues, with or without cell seeding). Such products generally have properties determined by the tissue source (i.e., tissue type and animal from which it originated) and the processing parameters used to produce the tissue products. Since tissue products are often used for surgical applications and/or tissue replacement or augmentation, the products should support tissue growth and regeneration and provide mechanical support to the defect in the implantation site.

In order to provide adequate support, tissue treatment devices must have sufficient strength to withstand the forces placed upon an implantation site. In addition, such devices must be properly secured to surrounding tissue and oriented to properly distribute applied forces to the devices without causing trauma to the implantation site.

Although effective for many indications, current tissue matrix products may develop laxity under prolonged loading. In addition, tissue matrices with different mechanical properties, e.g., higher strength (e.g., tensile, burst, tear) and/or reduced elongatability or higher elastic moduli may be desirable for some applications. Accordingly, the present disclosure provides improved tissue treatment products having synthetic elements within tissue matrices.

The present disclosure provides improved devices for treating anatomic defects. According to various embodiments, a device for treatment of an anatomic defect includes a flexible sheet of acellular tissue matrix having a first top surface and a second bottom surface. The device further includes one or more elongated synthetic elements, wherein when the flexible sheet of acellular tissue matrix lies flat, the one or more synthetic elements extend substantially parallel to the first top surface and the second bottom surface within the flexible sheet.

According to various embodiments, a device for treatment of an anatomic defect is provided. The device can be a flexible sheet of acellular tissue matrix having a first top surface and a second bottom surface. The device further includes one or more elongated synthetic elements extending through the flexible sheet such that the one or more synthetic elements extend substantially parallel to the first top surface and the second bottom surface within one or more portions of the flexible sheet. In addition, the one or more elongated synthetic elements are exposed on one or more portions of the first top surface of the device.

The present disclosure includes improved methods of producing a device for treatment of an anatomic defect. According to various embodiments, a method of producing a device for treatment of an anatomic defect includes selecting a flexible sheet of acellular tissue matrix having a first top surface and a second bottom surface. The method further includes inserting one or more elongated synthetic elements through at least a portion of an interior of the sheet of acellular tissue matrix such that when the flexible sheet of acellular tissue matrix lies flat, the one or more synthetic elements extend substantially parallel to the first top surface and the second bottom surface within the flexible sheet.

According to various embodiments, a method of producing a device for treatment of an anatomic defect includes selecting a flexible sheet of acellular tissue matrix having a first top surface and a second bottom surface. The method further includes inserting one or more elongated synthetic elements through at least a portion of an interior of the sheet of acellular tissue matrix such that the one or more synthetic elements extend substantially parallel to the first top surface and the second bottom surface within one or more portions of the flexible sheet, and are exposed on one or more portions of the first top surface of the flexible sheet.

The present disclosure includes also improved methods of treatment. According to various embodiments, the method includes selecting a treatment device and securing the treatment device within a selected anatomical site. The treatment device includes a flexible sheet of acellular tissue matrix having a first top surface and a second bottom surface and one or more synthetic elements, wherein when the flexible sheet of acellular tissue matrix lies flat, the one or more synthetic elements extend substantially parallel to the first top surface and the second bottom surface within the flexible sheet.

According to various embodiments, the method includes selecting a treatment device that includes a flexible sheet of acellular tissue matrix having a first top surface and a second bottom surface and one or more synthetic elements, such that the one or more synthetic elements extend substantially parallel to the first top surface and the second bottom surface within one or more portions of the flexible sheet and are exposed on one or more portions of the first top surface of the flexible sheet. The method further includes securing the flexible sheet of acellular tissue matrix and the one or more elongated synthetic elements within a selected anatomical site.

### DESCRIPTION OF THE DRAWINGS

FIG. 1A is a top perspective view of a treatment device produced according to various exemplary embodiments.

FIG. 1B is a side perspective view of the treatment device of FIG. 1A.

FIG. 1C is a top perspective view of a treatment device without elongated synthetic elements protruding from the tissue matrix.

FIG. 2A is a top perspective view of another treatment device produced according to various exemplary embodiments.

FIG. 2B is a side perspective view of the treatment device of FIG. 2A.

FIG. 3A is a top perspective view of another treatment device produced according to various exemplary embodiments.

FIG. 3B is a side perspective view of the treatment device of FIG. 3A.

FIG. 4 depicts an anatomical surgical site that may be treated with a treatment device according to various exemplary embodiments.

FIG. 5A illustrates an abdominal opening treated using a treatment device of the present disclosure attached with sutures.

FIG. 5B illustrates an abdominal opening treated using a treatment device of the present disclosure attached with barbs.

FIGS. 6A-6F illustrate fixation elements that may be incorporated with the disclosed devices.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Various human and animal tissues can be used to produce products for treating patients. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products can include, for example, acellular tissue matrices, tissue allografts or xenografts, and/or reconstituted tissues (i.e., at least partially decellularized tissues that have been seeded with cells to produce viable materials).

A variety of tissue products have been produced for treating soft and hard tissues. For example, ALLODERM® and STRATTICE™ (LIFECELL CORPORATION, Branchburg, N.J.) are two dermal acellular tissue matrices made from human and porcine dermis, respectively. Although such materials are very useful for treating certain types of conditions, materials having different biological and mechanical properties may be desirable for certain applications. For example, ALLODERM® and STRATTICE™ have been used to assist in treatment of structural defects and/or to provide support to tissues (e.g., for abdominal walls or in breast reconstruction), and their strength and biological properties make them well-suited for such uses. Although effective for many applications, changes to such tissue matrices can make them even better for certain uses, including additional complex abdominal wall repairs, high load applications, or other complex anatomic defects.

Accordingly, disclosed herein are tissue matrix products that can include embedded synthetic elements. The synthetic elements can reinforce the tissue matrix to provide improved structural features useful for a variety of applications. The reinforced tissue matrix can be configured to distribute at least a portion of forces to synthetic elements within the reinforced tissue matrix, thereby improving treatment for some load-bearing applications. The synthetic elements are embedded within at least a portion of the tissue matrix material, thereby reducing or preventing direct contact with tissues to prevent undesirable immunologic responses that can lead to scarring, adhesion to the synthetic elements, or undesirable immunologic response.

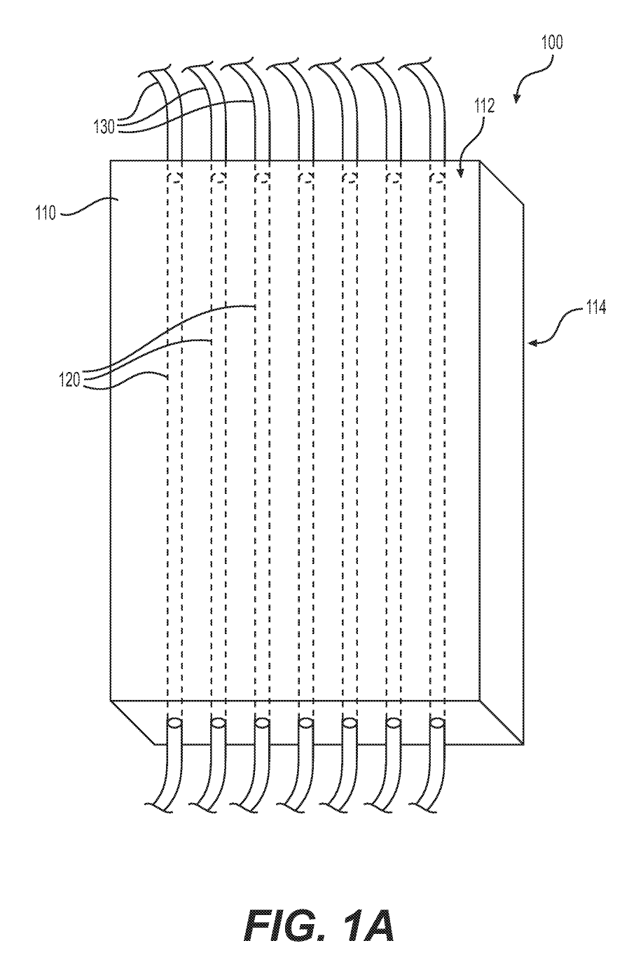

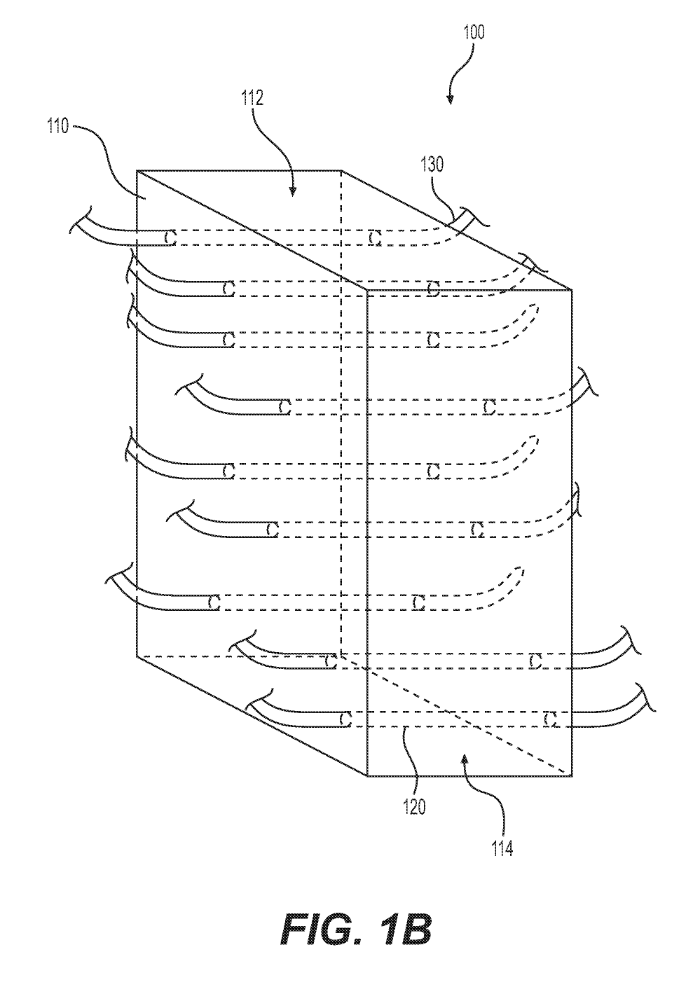

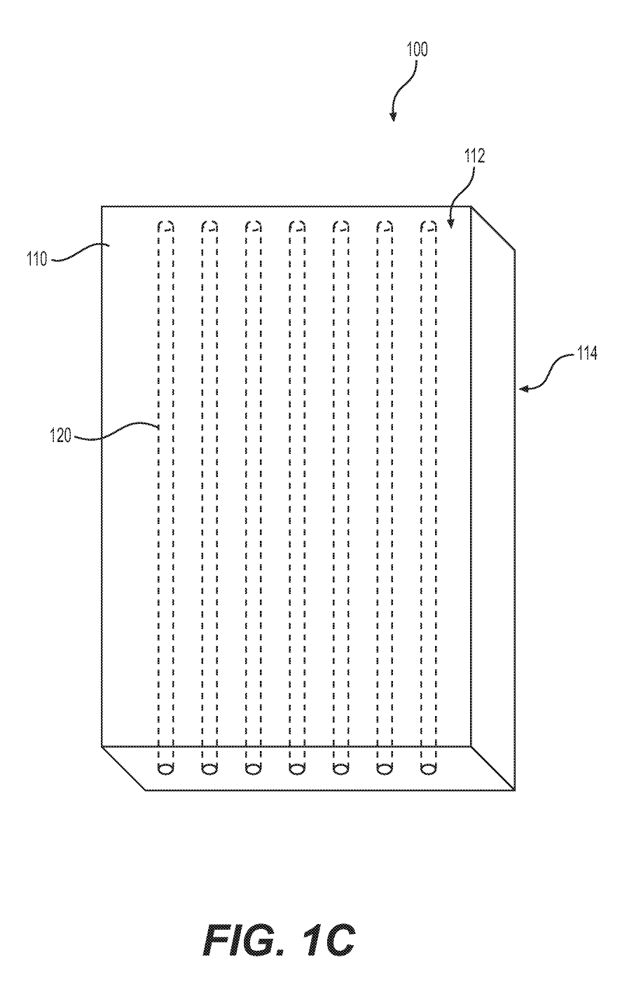

FIG. 1A is a top perspective view of a treatment device 100 according to various exemplary embodiments. The treatment device 100 can include a tissue matrix 110. The tissue matrix 110 may be in the form of a flexible sheet having a first top surface 112 and a second bottom surface 114 along with one or more elongated synthetic elements 120, wherein when the flexible sheet of tissue matrix 110 lies flat, the one or more elongated synthetic elements 120 extend substantially parallel to the first top surface and the second bottom surface within the tissue matrix 110.

The tissue matrix 110 is illustrated as a two-dimensional view of a flexible sheet of material. Accordingly, it should be appreciated that the flexible sheet will have a length and width, and a thickness. The length, width, and thickness can be selected based on the desired surgical indication, e.g., to provide a sufficient surface area (measured in terms of the length and width) and structural stability (e.g., based on strength, tensile properties, suture retention, burst strength, etc.). The flexible sheet 110 may range in width between 10 cm and 30 cm, between 10 cm and 25 cm, between 20 cm and 25 cm, or any ranges in between. In addition the flexible sheet 110 may range in length between 10 cm and 30 cm, between 15 cm and 30 cm, or between 20 cm and 25 cm, or any ranges in between. For dermal tissue matrix materials, the thickness can vary, but may be between, for example, 0.75 mm to 4 mm, 0.75 mm to 1.25 mm, or 1.05 mm to 1.55 mm.

The tissue matrix 110 may be composed of a variety of suitable materials. For example, in some embodiments, the tissue matrix 110 is produced from tissue derived from dermal, fascia, adipose, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, dermal tissue, muscle, and intestinal tissue. The tissue may be selected from human or porcine tissue sources.

In some embodiments, the tissue matrix 110 is an acellular tissue matrix. In further embodiments, the tissue matrix 110 is dermal acellular tissue matrix. In further embodiments, the dermal acellular tissue may be selected from human or porcine tissue sources.

In addition, if extracellular matrix materials are used, the materials can be further processed to control desired properties, including porosity, mechanical properties (strength, compressibility, elasticity), and/or biological properties (e.g., enzyme susceptibility, in vivo degradation rate, and/or ability to support in growth and tissue regeneration for intended treatment sites). Such processing can include micronizing, re-suspension, cross-linking, defatting, decellularizing, and/or lyophilizing. Exemplary processing techniques are described further below.

As noted above, the device 100 further includes one or more elongated synthetic elements 120. The elongated synthetic elements 120 may be composed of any material capable of withstanding the various mechanical demands exerted upon the treatment device 100 in the implantation site. The various mechanical demands may include, but are not limited to, compressive strength, durability, elasticity, flexibility, impact resistance, shear modulus, shear strength, stiffness, tensile strength, and Young's modulus. For example, if the treatment device 100 is implanted over a midline incision of the abdomen, the elongated synthetic elements 120 can be capable of withstanding at least a primary force exerted upon the midline.

In some embodiments, the elongated synthetic elements 120 are non-resorbable. Treatment devices 100 including non-resorbable elongated synthetic elements 120 can be permanently left in an implantation site. Non-resorbable materials would be desirable in certain embodiments because resorbable materials lose strength over time as the body absorbs the material. In some embodiments, resorbable elongated synthetic elements 120 are desirable to create non-permanent treatment devices 100.

The elongated synthetic elements 120 may be composed of polymeric materials. In some embodiments, the elongated synthetic elements 120 comprise at least one of at least one of polypropylene, polytetrafluoroethylene, polyester, terephthalate, polyglycolide, or poly-4-hydroxybutyrate. In some embodiments, the elongated synthetic elements 120 may be composed of non-resorbable polymeric materials.

Each elongated synthetic element 120 has a flexible three-dimensional structure comprising a length dimension, a width dimension, and a height dimension. In some embodiments, the length is at least about one hundred times larger than the height and width. In some embodiments, the length is at least about five hundred times larger than the height and width. In some embodiments, the length is at least about one thousand times larger than the height and width. In some embodiments, the length is at least about two thousand times larger than the height and width.

In some embodiments, the elongated synthetic elements 120 pass through and protrude from at least one end of the tissue matrix 110. In some embodiments, the elongated synthetic elements 120 are surrounded by the tissue matrix material of the tissue matrix 110. The elongated synthetic elements 120 may be inserted through the tissue matrix 110 and cut or tied at each end of the tissue matrix 110. In some embodiments, the tissue matrix 110 is formed around the elongated synthetic elements 120. In further embodiments, the elongated synthetic elements 120 do not protrude out of the tissue matrix 110.

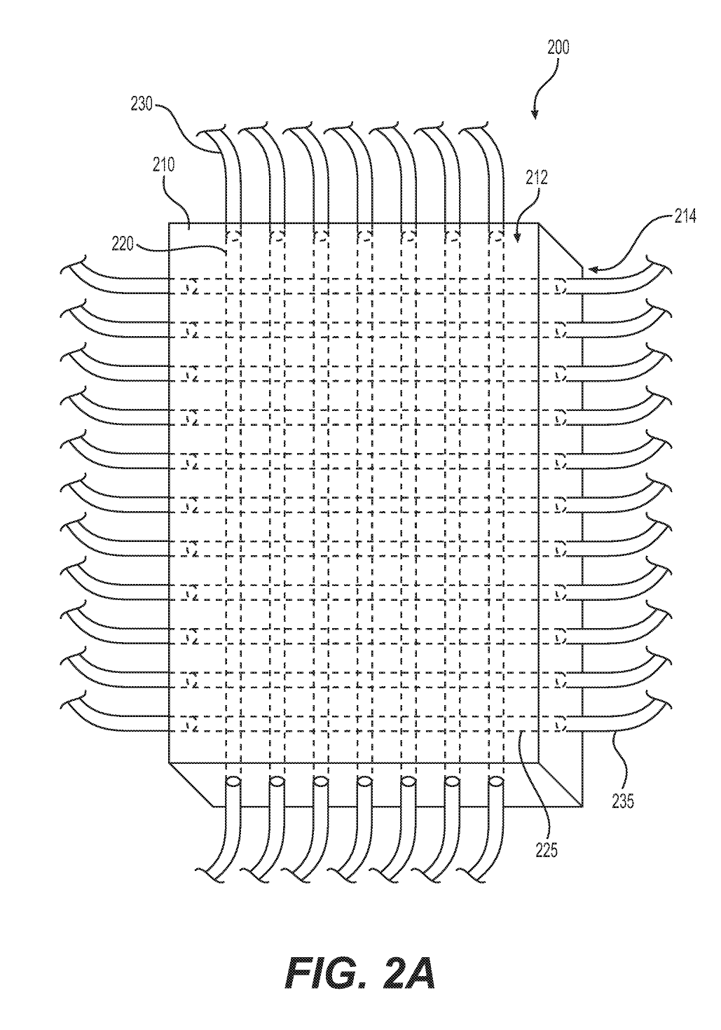

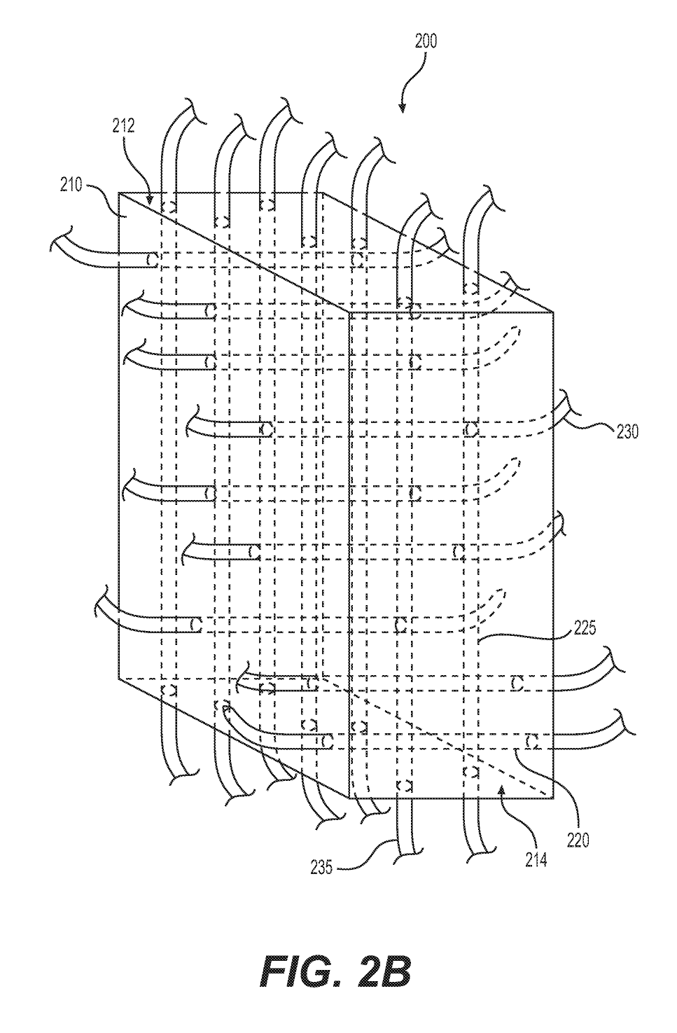

The elongated synthetic elements 120 may be oriented within the tissue matrix 110 according to an intended use of the treatment device 100 or according to the preference or a surgeon, physician, nurse, or other medical professional. The elongated synthetic elements 120 may be substantially parallel to the outer surfaces of the flexible sheet. In some embodiments, the elongated synthetic elements 120 may be at an angle relative to the first top surface 112 and the second bottom surface 114 of the tissue matrix 110. In some embodiments, the elongated synthetic elements 120 are substantially parallel with one another. In some embodiments, the elongated synthetic elements 120 may be evenly distributed throughout the flexible sheet. In some embodiments, a first group of elongated synthetic elements within the tissue matrix 110 extends perpendicularly to a second group of elongated synthetic elements within the tissue matrix 110, as depicted in FIGS. 2A and 2B described in further detail below.

In some embodiments, the elongated synthetic elements 120 are sutures. The sutures may be monofilament, braided, braided and coated, or a combination thereof. In some embodiments, the sutures are non-resorbable.

The treatment device 100 further includes at least one fixation element 130 configured to attach the treatment device 100 to tissue. The fixation elements 130 may be barbs, flaps, tacks, tabs, hooks, adhesive-covered elements, or a combination thereof. In some embodiments, the fixation elements 130 are barbed sutures.

The fixation elements 130 may be composed of biocompatible materials. In some embodiments, the fixation elements 130 are composed of the same material as the elongated synthetic elements 120. In some embodiments, the fixation elements 130 are composed of synthetic materials. In some embodiments, the fixation elements 130 are non-resorbable. In some embodiments, the fixation elements 130 comprise at least one of polypropylene, polytetrafluoroethylene, polyester, terephthalate, polyglycolide, or poly-4-hydroxybutyrate.

The fixation elements 130 may be separate components of the treatment device 100 or may be extensions of the elongated synthetic elements 120. For example, the elongated synthetic elements 120 may extend outward from one or more surfaces of the tissue matrix 110 to form the fixation elements 130. As a further example, each fixation element 130 is a continuous extension of an elongated synthetic element 120. In other words, a treatment device 100 may include at least one unitary synthetic element of one elongated synthetic element 120 with a fixation element 130 on one or both ends of the elongated synthetic element 120. In this example, the elongated synthetic elements 120 and fixation elements 130 may be sutures.

In some embodiments, the fixation elements 130 are attached to the elongated synthetic elements 120. The fixation elements 130 may be attached to one or both exposed ends of each elongated synthetic element 120. In some embodiments, fixation elements 130 are attached to some, but not all of the elongated synthetic elements 120. The fixation elements 130 may be attached to the elongated synthetic elements 120 either before or after the elongated synthetic elements 120 are inserted through the tissue matrix 110, or the tissue matrix 110 is formed around the elongated synthetic elements 120. In some embodiments, during insertion, the fixation elements 130 pierce through the tissue matrix 110 to allow for insertion of the elongated synthetic elements 120.

The treatment device 100 may be produced in various shapes and sizes. For example, the treatment device 100 may be shaped to conform to an implantation site, fill an implantation site, surround an implantation site, or cover an implantation site. Although described as a sheet, the tissue matrix 110 could be in the form of a cylinder, box, sphere, or irregular shape.

A treatment device 100 shaped as a sheet may be used to surround or cover an implantation site. For example, the sheet may be wrapped around a tendon or ligament. The sheet may also cover an incision produced by surgery or other trauma. The sheet may cover any anatomical defect. In some embodiments, the sheet is oriented to withstand loads that will be forced upon the implantation site.

A treatment device 100 shaped as a cylinder may be used to surround or fill an implantation site. For example, the cylinder may be placed around a reconstructed implantation site such as a tendon or ligament. The cylinder may also be placed around an organ. In some embodiments, the cylinder fills in a cylindrical implantation site.

A treatment device 100 shaped as a box may be used to fill an implantation site. For example, the box may be used to fill an implantation site that features an area where tissue was removed by surgery or trauma.

A treatment device 100 shaped as a sphere or an irregular shape may be used to fill or conform to an implantation site. For example, one or more spheres may be inserted into an anatomical defect to fill the defect. The one or more spheres may be mixed with one or more irregularly shaped devices.

The treatment device 100 may be placed at any orientation in or around an implantation site. However, certain orientations of the treatment device 100, specifically the orientations of the elongated synthetic elements 120 within the tissue matrix 110, are more effective at receiving forces upon the implantation site. In some embodiments, elongated synthetic elements 120 oriented substantially perpendicular to a tendon, ligament, incision, or other traumatized tissue receive and protect the traumatized tissue from a greater force than elongated synthetic elements 120 oriented substantially parallel to the traumatized tissue. In some embodiments, the treatment device 100 is oriented such that a majority of the elongated synthetic elements 120 within the tissue matrix 110 are substantially perpendicular to the traumatized tissue of an implantation site. The orientation of the elongated synthetic elements 120 may also be determined by an expressed preference of a user of the treatment device 100.

As a non-limiting example, the treatment device 100 may be implanted along a midline incision of an abdomen after surgery. The treatment device 100 is implanted such that the elongated synthetic elements 120 are oriented substantially perpendicular to the incision. In a perpendicular orientation, the elongated synthetic elements 120 will receive a minority, a majority, or all of the force directed to the area of the incision. Once the treatment device 100 is correctly oriented, the treatment device 100 is sutured to the implantation site with the fixation elements 130.

FIG. 1B is a side perspective view of the treatment device 100 of FIG. 1A. FIG. 1C is a top perspective view of a treatment device 100 without elongated synthetic elements 120 protruding from the tissue matrix 110. The elongated synthetic elements 120 in FIG. 1C extend through the tissue matrix 110 but do not protrude out from the matrix. As can be seen in FIGS. 1A, 1B, and 1C, the elongated synthetic elements 120 are substantially parallel with one another within the tissue matrix 110.

Examples of various combinations of materials are described below. It will be understood that some of the materials listed can be interchanged in the various embodiments without departing from the intended scope of the invention.

The treatment device 100 can be modified to include a second group of elongated synthetic elements. FIG. 2A is a top perspective view of a treatment device 200 produced according to various exemplary embodiments. Similar to treatment device 100 described with respect to FIGS. 1A, 1B, and 1C, treatment device 200 includes a tissue matrix 210 and a first group of elongated synthetic elements 220 that may include fixation elements 230.

As shown, the treatment device 200 includes a tissue matrix 210, a first group of elongated synthetic elements 220, and a second group of elongated synthetic elements 225. In these embodiments, the first group of elongated synthetic elements 220 is aligned perpendicularly to the second group of elongated synthetic elements 225. The second group of elongated synthetic elements 225 may include fixation elements 235 as described above.

FIG. 2B is a side perspective view of the treatment device of FIG. 2A. As can be seen in FIGS. 2A and 2B, the elongated synthetic elements within a group 220 or 225 are substantially parallel to one another. In some embodiments, the elongated synthetic elements 220 and 225 extend through the tissue matrix 210 along a direction perpendicular to a primary load that would be applied to the treatment device 200. In some embodiments, a portion of the elongated synthetic elements 220 and 225 extend throughout the tissue matrix 210 along a direction perpendicular to a primary load that would be applied to the treatment device 200. For example, if the treatment device 200 is implanted over a midline incision of the abdomen, the elongated synthetic elements 220 and 225 can be capable of withstanding at least a primary force exerted upon the midline.

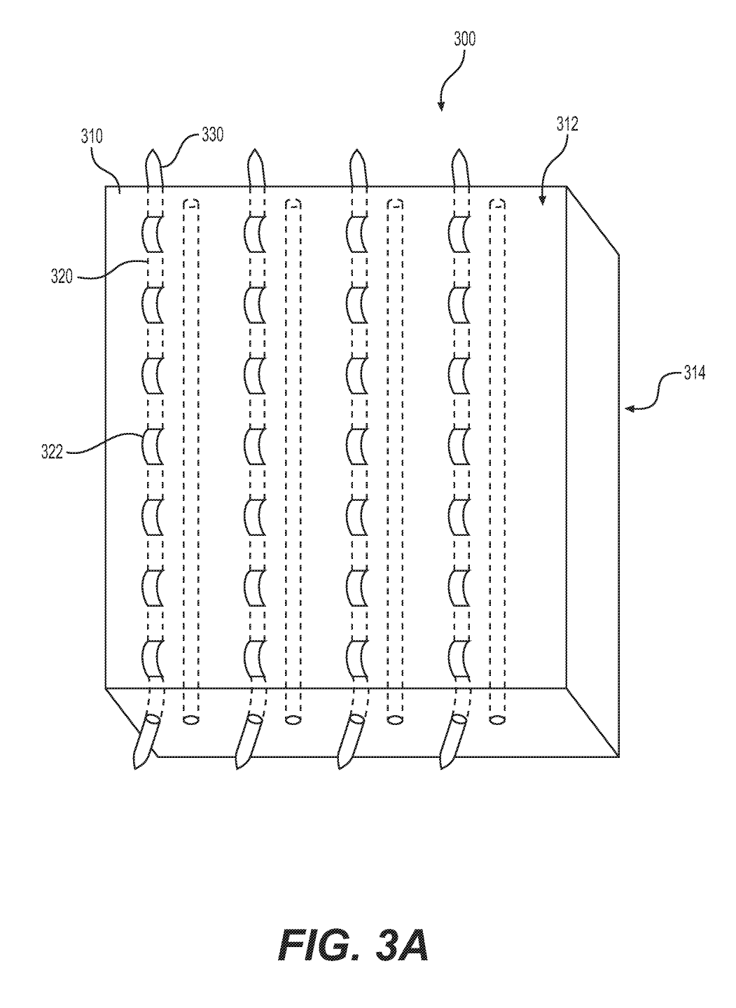

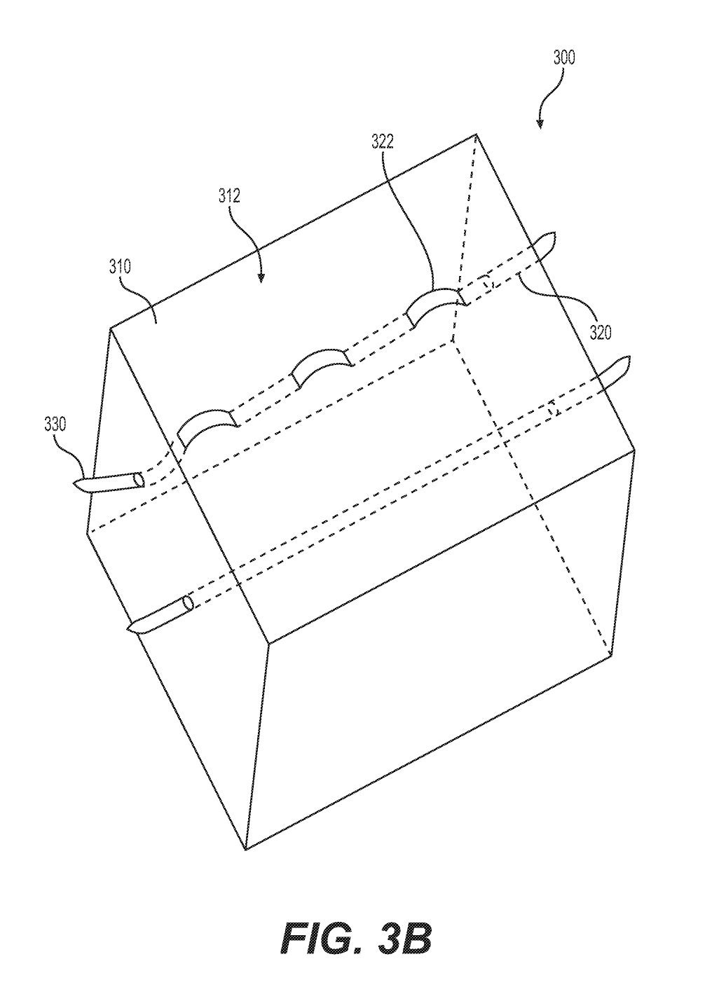

The treatment device can be modified to expose the one or more elongated synthetic elements on one or more portions of the top surface of the tissue matrix. FIG. 3A is a top perspective view of another treatment device 300 produced according to various exemplary embodiments. Similar to treatment device 100 described above with respect to FIGS. 1A, 1B, and 1C, treatment device 300 includes a tissue matrix 310 and a first group of elongated synthetic elements 320 that may include fixation elements 330.

The elongated synthetic elements 320 may be oriented within the tissue matrix 310 according to an intended use of the treatment device 300 or according to the preference or a surgeon, physician, nurse, or other medical professional. In some embodiments, the one or more synthetic elements 320 extend through the tissue matrix 310 such that at least one or more of the elongated synthetic elements 322 extend substantially parallel to the first top surface 312 and the second bottom surface 314 of one or more portions of the tissue matrix 310, and are exposed on one or more portions of the first top surface 312 of the tissue matrix 310. At least one or more elongated synthetic elements 320 may extend through the tissue matrix 310 as described above with respect to FIG. 1 without partial exposure through the top surface 312. In some embodiments, one or more portions of the elongated synthetic elements 320 may be at an angle relative to the first top surface 312 and the second bottom surface 314 of the tissue matrix 310 and one or more portions of the elongated synthetic elements 320 are exposed on one or more portions of the first top surface 312 of the tissue matrix 310.

FIG. 3B is a side perspective view of the treatment device of FIG. 3A. As can be seen in FIGS. 3A and 3B, the exposed elongated synthetic elements 322 are substantially parallel with one another. In some embodiments, the exposed elongated synthetic elements 322 extend throughout the tissue matrix 310 along a direction perpendicular to a primary load that would be applied to the treatment device 300. In some embodiments, a portion of the exposed elongated synthetic elements 322 extend throughout the tissue matrix 310 along a direction perpendicular to a primary load that would be applied to the treatment device 300. For example, if the treatment device 300 is implanted over a midline incision of the abdomen, the elongated synthetic elements 320 and exposed elongated synthetic elements 322 can be capable of withstanding at least a primary force exerted upon the midline. At least one or more elongated synthetic elements 320 may extend through the tissue matrix 310 as described above with respect to FIG. 1 without exposure through the top surface 312.

After selecting the tissue matrix, one or more elongated synthetic elements may be inserted through at least a portion of an interior of the tissue matrix. In some embodiments, the one or more elongated synthetic elements are passed through a flexible sheet of tissue matrix such that when the flexible sheet of acellular tissue matrix lies flat, the one or more synthetic elements extend substantially parallel to the first top surface and the second bottom surface within the flexible sheet. In some embodiments, the one or more elongated synthetic elements are inserted through at least a portion of an interior of the sheet of acellular tissue matrix such that the one or more synthetic elements partially extend substantially parallel to the first top surface and the second bottom surface within the flexible sheet, and are partially exposed on the first top surface of the flexible sheet. In some embodiments, the one or more elongated synthetic elements are inserted such that they are substantially parallel with one another.

In some embodiments, the selected tissue matrix is formed around the one or more elongated synthetic elements. In some embodiments, the tissue matrix is formed around a first group of elongated synthetic elements and a second group of synthetic elements is inserted through the formed tissue matrix.

The devices described above further include one or more fixation elements. The fixation elements may extend from the one or more elongated synthetic elements or may be attached to the one or more elongated synthetic elements after the one or more elongated synthetic elements are joined with the tissue matrix. In some embodiments, the fixation elements are utilized to pierce through the tissue matrix and insert the one or more elongated synthetic elements through the tissue matrix.

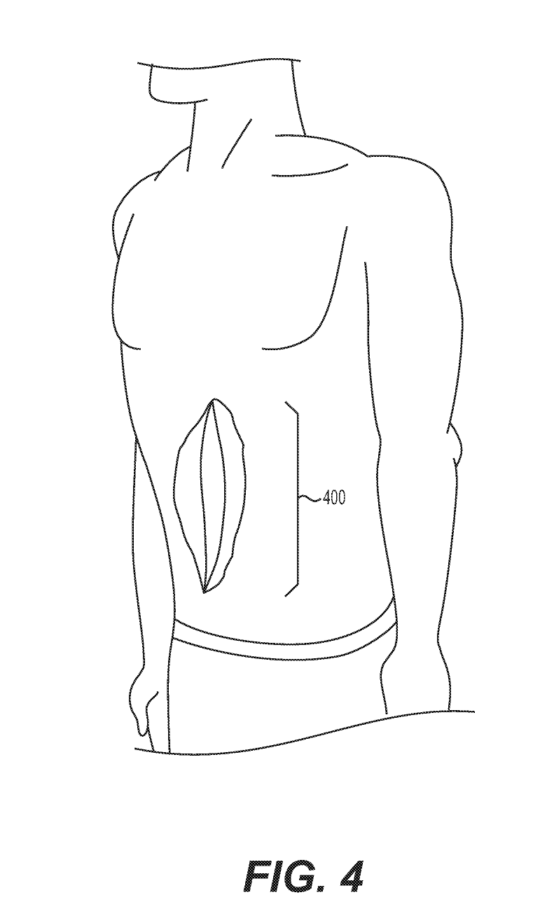

FIG. 4 depicts an anatomical surgical site 400 that may be treated with a treatment device according to various exemplary embodiments. The surgical site 400 depicted in FIG. 4 is an abdominal site. However, the above described devices may be used to treat surgical sites in other anatomic locations of the body. In some embodiments, the surgical site 400 is along a midline incision of an abdominal wall. In further embodiments, the anatomical site 400 is a tendon along the midline of the abdomen. After selecting a treatment device, a user of the device secures the device within the anatomical surgical site 400.

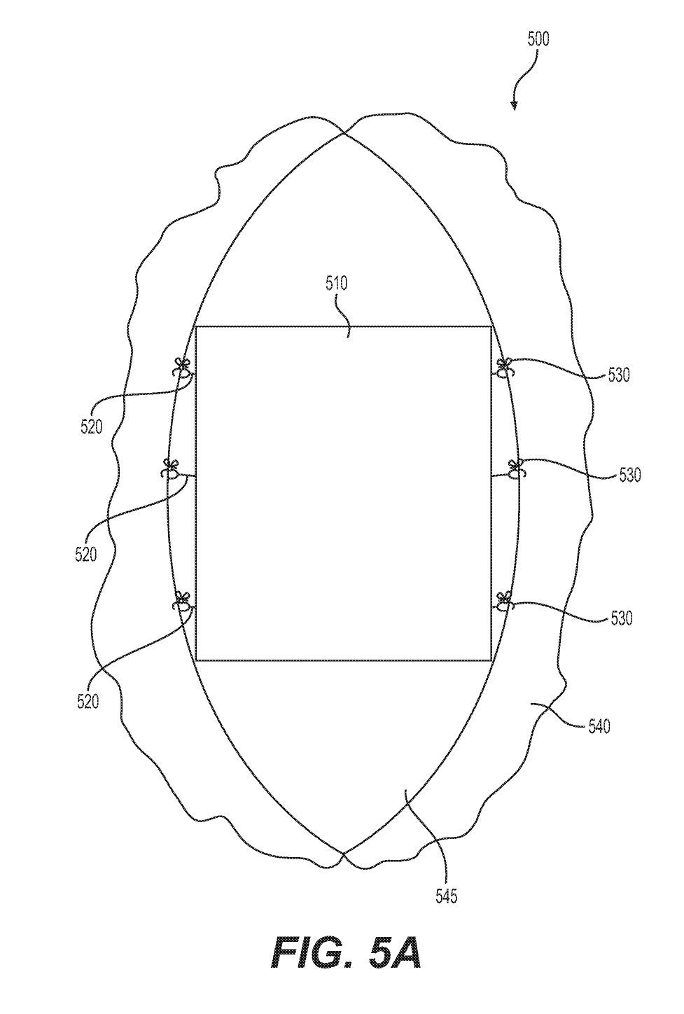

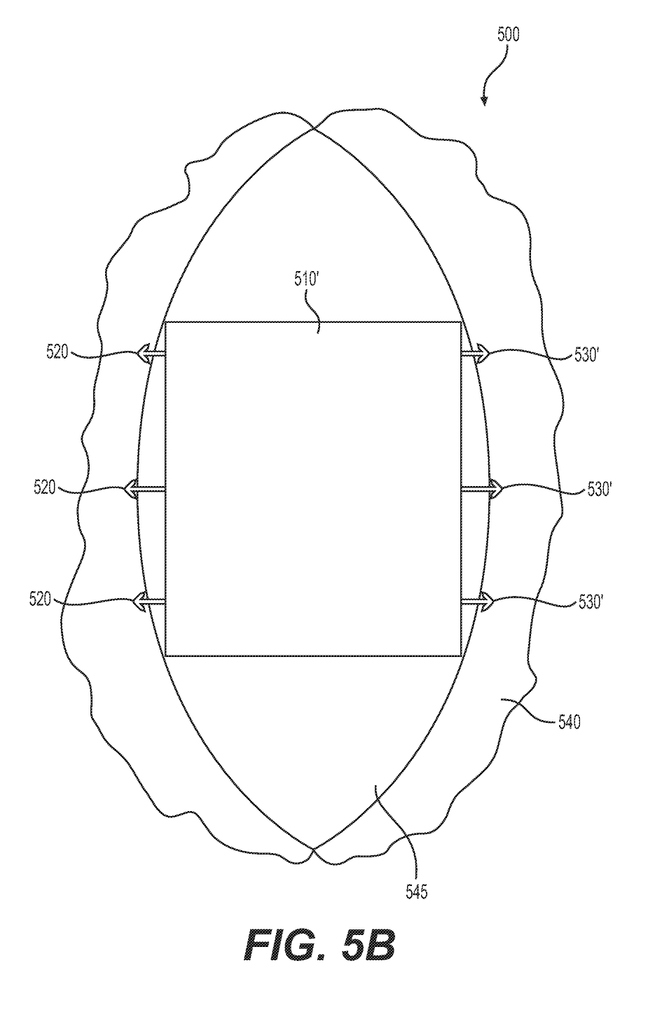

FIG. 5A illustrates an abdominal opening 500 treated using a treatment device 510 of the present disclosure with elongated elements 520 attached with sutures 530. The device 510 is secured within the surgical site 500. The device 510 may be secured to dermal tissue 540, connective and/or muscle tissue 545, or a combination thereof, with fixation elements 530. In the depicted embodiment, the fixation elements 530 are sutures 530. The treatment device 510 is secured such that the one or more elongated synthetic elements 520 within the device 510 are aligned substantially perpendicular to the incision of the abdominal wall. In some embodiments, the treatment device 510 is secured such that a majority of the one or more elongated synthetic elements 520 within the device 510 are aligned substantially perpendicular to the incision of the abdominal wall.

In some embodiments, one or more elongated synthetic elements 520 partially extend substantially parallel to the first top surface and the second bottom surface within the treatment device 510 and are partially exposed on the first top surface of the treatment device 510. The treatment device 510 is secured within the surgical site 500 such that the bottom surface is adjacent to the exposed connective and/or muscular tissue 545 within the surgical site 500 and the top surface faces away from the exposed connective and/or muscular tissue 545 within the surgical site 500. Orienting the device 510 so that the top surface including exposed elongated synthetic elements faces away from the connective and/or muscle tissue 545 prevents adhesion between the exposed elongated synthetic elements and the exposed connective and/or muscular tissue 545 within the surgical site 500.

FIG. 5B illustrates an abdominal opening 500 treated using a treatment device 510′ of the present disclosure with elongated elements 520 attached with barbs 530′. The device 510′ is secured within the surgical site 500. The device 510′ is secured to dermal tissue 540, connective and/or muscle tissue 545, or a combination thereof, with fixation elements 530′. In the depicted embodiment, the fixation elements 530′ are barbs 530′. The treatment device 510′ is secured in a similar manner to device 510.

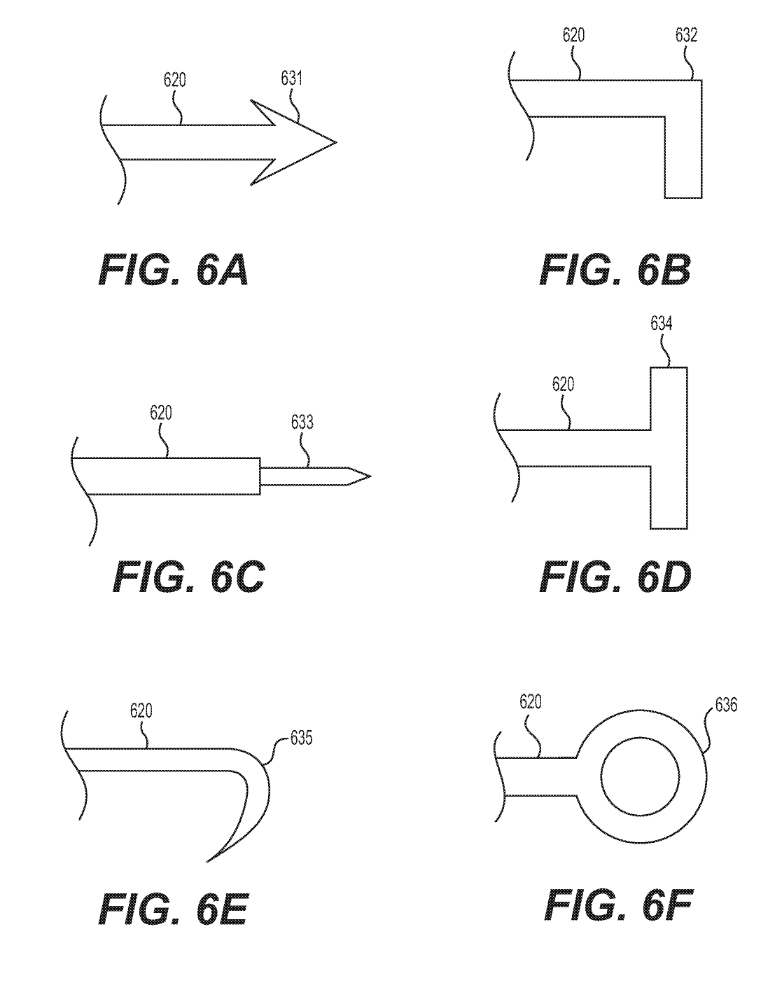

FIGS. 6A-6F are enlarged views of the fixation elements 631-636 of the disclosed treatment devices. The fixation elements 631-636 may be barbs, flaps, tacks, tabs, hooks, loops, adhesive-covered elements, or a combination thereof. The fixation elements 631-636 are similar to fixation elements 130, 230, 330, and 530 shown in the Figures described above. The fixation elements 631-636 are attached to elongated synthetic elements 620. The elongated synthetic elements 620 are similar to the elongated synthetic elements 120, 220, 320, and 520 shown in the Figures described above that are attached to fixation elements 130, 230, 330, and 530. In some embodiments, the fixation elements are barbed sutures.

FIG. 6A depicts a barb 631 attached to an elongated synthetic element 620. The narrow distal end of the barb 631 is configured to puncture tissue surrounding the elongated synthetic element 620. In some embodiments, the tissue is connective tissue such as fascia of the abdominal wall of a patient. After the barb 631 punctures tissue, the barb 631 may be passed through the puncture site in the tissue. Then the flared proximal end of the barb 631 rests against the puncture site within the tissue, preventing removal of the barb 631 from the tissue. In some embodiments, an adhesive is pre-applied to the barb 631 to further secure the barb 631 to the tissue.

FIG. 6B depicts a flap 632 attached to an elongated synthetic element 620. The flap 632 is configured to enter an opening in tissue surrounding the elongated synthetic element 620. In some embodiments, the tissue is connective tissue such as fascia of the abdominal wall of a patient. The opening may be a slit, tear, hole, or other suitable entry portal to receive the flap 632. The opening may be created by a medical professional implanted a treatment device that includes the elongated synthetic elements 620 and flap 632. After insertion into the tissue, the flap 632 may be folded over to secure the elongated synthetic element 620. In some embodiments, an adhesive is pre-applied to the flap 632 to further secure the flap 632 to the tissue.

FIG. 6C depicts a tack 633 attached to an elongated synthetic element 620. The distal end of the tack 633 is configured to puncture tissue surrounding the elongated synthetic element 620. In some embodiments, the tissue is connective tissue such as fascia of the abdominal wall of a patient. The tack 633 is inserted in the tissue to securely remain in place when forces are exerted upon the puncture site. In some embodiments, an adhesive is pre-applied to the tack 633 to further secure the tack 633 to the tissue.

FIG. 6D depicts a tab 634 attached to an elongated synthetic element 620. The tab 634 is configured to enter an opening in tissue surrounding the elongated synthetic element 620. In some embodiments, the tissue is connective tissue such as fascia of the abdominal wall of a patient. The opening may be a slit, tear, hole, or other suitable entry portal to receive the tab 634. The opening may be created by a medical professional implanted a treatment device that includes the elongated synthetic elements 620 and tab 634. After insertion into the tissue, the tab 634 may be folded over to secure the elongated synthetic element 620. In some embodiments, an adhesive is pre-applied to the tab 634 to further secure the tab 634 to the tissue.

FIG. 6E depicts a hook 635 attached to an elongated synthetic element 620. The distal end of the hook 635 is configured to puncture tissue surrounding the elongated synthetic element 620. In some embodiments, the tissue is connective tissue such as fascia of the abdominal wall of a patient. The hook 635 may be fully inserted into the tissue to secure the elongated synthetic element 620. In some embodiments, an adhesive is pre-applied to the hook 635 to further secure the hook 635 to the tissue.

FIG. 6F depicts a loop 636 attached to an elongated synthetic element 620. The loop 636 is configured to receive sutures or other fixation devices (e.g., such as a barb 631, flap 632, tack 633, tab 634, or hook 635) for securing the elongated synthetic element 620 to surrounding tissue. In some embodiments, the tissue is connective tissue such as fascia of the abdominal wall of a patient. In some embodiments, at least one suture is passed through multiple loops 636 to secure multiple elongated synthetic elements 620.

What is claimed is:

1. A device for treatment of an anatomic defect, comprising:
a flexible sheet of acellular tissue matrix having a first top surface and a second bottom surface; and
one or more elongated synthetic elements, wherein when the flexible sheet of acellular tissue matrix lies flat, the one or more synthetic elements extend substantially parallel to the first top surface and the second bottom surface within the flexible sheet.

2. The device of claim 1, wherein each elongated synthetic element has a flexible three-dimensional structure comprising a length dimension, a width dimension, and a height dimension, and wherein one dimension is at least about one hundred times larger than the other two dimensions.

3. The device of claim 1, wherein the one or more elongated synthetic elements are substantially parallel with one another.

4. The device of claim 1, wherein the one or more elongated elements include a first group of elongated synthetic elements and a second group of elongated synthetic elements, and wherein the first group of elongated synthetic elements are aligned perpendicularly to the second group of elongated synthetic elements.

5. The device of claim 1, further comprising one or more fixation elements.

6. The device of claim 5, wherein the one or more elongated synthetic elements further comprise the one or more fixation elements.

7. The device of claim 5, wherein the one or more fixation elements extend from the one or more elongated synthetic elements.

8. The device of claim 5, wherein the one or more fixation elements comprise at least one of barbs, tacks, tabs, hooks, flaps, or loops.

9. The device of claim 1, wherein the one or more elongated synthetic elements are selected from the group comprising monofilament materials, braided materials, braided and coated materials, or a combination thereof.

10. The device of claim 1, wherein the one or more elongated synthetic elements extend through the flexible sheet such that the one or more synthetic elements extend substantially parallel to the first top surface and the second bottom surface within one or more portions of the flexible sheet, and are exposed on one or more portions of the first top surface of the device.

11. A method of producing a device for treatment of an anatomic defect, comprising:
selecting a flexible sheet of acellular tissue matrix having a first top surface and a second bottom surface; and
inserting one or more elongated synthetic elements through at least a portion of an interior of the sheet of acellular tissue matrix such that when the flexible sheet of acellular tissue matrix lies flat, the one or more synthetic elements extend substantially parallel to the first top surface and the second bottom surface within the flexible sheet.

12. The method of claim 11, wherein each elongated synthetic element has a flexible three-dimensional structure comprising a length dimension, a width dimension, and a height dimension, and wherein one dimension is at least one about hundred larger than the other two dimensions.

13. The method of claim 11, wherein the one or more elongated synthetic elements are inserted such that they are substantially parallel with one another.

14. The method of claim 11, wherein the one or more elongated elements includes a first group of elongated synthetic elements and a second group of elongated synthetic elements, and wherein the first group of elongated synthetic elements are aligned perpendicular to the second group of elongated synthetic elements.

15. The method of claim 11, further comprising securing at least some of the one or more elongated synthetic elements to tissue using one or more fixation elements attached to the one or more elongated synthetic elements.

16. The method of claim 15, wherein the one or more fixation elements extend from the one or more elongated synthetic elements.

17. The method of claim 15, wherein the one or more fixation elements comprise at least one of barbs, tacks, tabs, hooks, flaps, or loops.

18. The method of claim 11, wherein the one or more elongated synthetic elements are inserted such that the one or more synthetic elements extend substantially parallel to the first top surface and the second bottom surface within one or more portions of the flexible sheet, and are exposed on one or more portions of the first top surface of the flexible sheet.

19. A method of treatment, comprising:
selecting a treatment device comprising:
a flexible sheet of acellular tissue matrix having a first top surface and a second bottom surface;
and one or more synthetic elements, wherein when the flexible sheet of acellular tissue matrix lies flat, the one or more synthetic elements extend substantially parallel to the first top surface and the second bottom surface within the flexible sheet; and
securing the treatment device within a selected anatomical site.

20. The method of claim 19, wherein the treatment device is secured such that the one or more elongated synthetic elements are aligned substantially perpendicular to an incision of an abdominal wall.
